(19) United States
(12) Patent Application Publication
(10) Pub. No.: US 2016/0022385 A1
(43) Pub. Date: Jan. 28, 2016

(54) DENTAL IMPLANT SYSTEM AND METHOD OF USE

(71) Applicant: Kun Zhao, Quincy, MA (US)

(72) Inventor: Kun Zhao, Quincy, MA (US)

(21) Appl. No.: 14/810,118

(22) Filed: Jul. 27, 2015

Related U.S. Application Data
(60) Provisional application No. 62/029,722, filed on Jul. 28, 2014.

(51) Int. Cl. A61C 8/00; A61C 13/00; A61C 9/00; A61B 6/14; A61C 13/30; A61C 13/08

(52) U.S. Cl. CPC A61C 8/0031; A61C 13/30; A61C 13/081; A61C 9/004; A61B 6/14; A61C 13/0004; A61C 8/0012

(57) ABSTRACT

A dental implant system including a prosthodontic base having first and second opposing surfaces, the first surface configured to conform to and abut an edentulous ridge of a patient, the second surface having a post extending outwardly therefrom, the prosthodontic base defining at least one hole for receiving an implant. The system further including a restoration having at least one artificial tooth, the restoration defining an opening to receive the post, the restoration being attachable to the post. The system further includes an implant having a head and a body, the body of the implant insertable through the hole in the prosthodontic base and into a bone of the patient for attaching the prosthodontic base to the edentulous ridge. The dental implant system providing for efficient, minimum invasive and permanent teeth replacement for the patient. A method of providing one or more teeth replacement for a patient is also disclosed.

### CROSS-REFERENCE TO RELATED APPLICATIONS

This application claims the benefit under 35 U.S.C. §119(e) of U.S. Provisional Application No. 62/029,722 filed Jul. 28, 2014, the entire disclosure of which is hereby incorporated by reference.

### FIELD OF THE INVENTION

The present disclosure relates generally to a dental implant system and method of use. More particularly, the disclosure relates to a dental implant system including a prosthodontic base, an implant and a restoration. Also disclosed is a method of application of the dental implant system.

### BACKGROUND OF THE INVENTION

The statements in this section merely provide background information related to the present disclosure and may not constitute prior art.

Missing tooth/teeth is a common problem that affects the oral health and general health of many people. A person can suffer from a single tooth missing, multiple teeth missing or even fully edentulous. Without appropriate replacement, a missing tooth/teeth can have many negative impacts on the dentition, occlusion, temporomandibular joint function, maxilla and mandible bone structure, as well as a person's overall general health.

Currently, there are many methods to replace missing tooth/teeth, ranging from removable partial restoration, full restoration, fixed bridge, to implant supported crown, bridge and overdenture.

Dental implants are typically more advanced methods for replacing one tooth and/or multiple teeth. One benefit of dental implants is the tendency of the dental implant to slow or stop the atrophic process of the jaw bone after a tooth or teeth are missing. Dental implants may be categorized differently, but for common root shaped endosteal implants, there are often two categories, namely, regular implant or mini implant based on size. For example, regular implants have a diameter larger than 3.0 mm (3.2 mm or larger in most cases) while mini implants have a diameter less than 3.0 mm. (1.8 to 2.9 mm, in most cases). Both regular and mini implants can be used to replace a singular tooth, multiple teeth or even a full dentition. However, mini implants are typically used with singular tooth replacements and overdenture.

It is an object of the present teachings to provide a novel dental implant system and method of use that overcomes the shortcomings of prior art restorations and methods.

### SUMMARY OF THE INVENTION

This section includes a general summary of the disclosure and does not provide a comprehensive description or include full scope or all the features of the subject matter disclosed.

The present teachings provide a dental implant system including a prosthodontic base having first and second opposing surfaces, the first surface configured to conform to and abut an edentulous ridge of a patient, the second surface having at least one post extending outwardly therefrom, the prosthodontic base defining at least one hole for receiving an implant. The dental implant system further includes a restoration having at least one artificial tooth, the restoration defining at least one opening to receive the post, wherein the restoration is attachable to the post(s) for fixing the restoration to the prosthodontic base. An implant having a head, a neck and a body is also provided. The body of the implant being insertable through the hole in the prosthodontic base and into a jaw bone of the patient for attaching the prosthodontic base to the edentulous ridge, the head being larger than a diameter of the hole in the prosthodontic base for securing the prosthodontic base adjacent the edentulous ridge of the patient. The dental implant system provides for efficient, minimum invasive and permanent replacement teeth for the patient.

In another aspect, the dental implant system includes the first surface of the prosthodontic base being formed based on an impression (conventional or digital impression) of the edentulous ridge.

In another aspect, the dental implant system includes a nail shaped implant for securing the prosthodontic base to the edentulous ridge of the patient. The implant may be a mini implant having a diameter less than about 3.0 mm. In one embodiment, the implant is configured to be installed through the prosthodontic base via an automatic device such as a nail gun. The implant being nail-shaped and configured for attachment to the edentulous ridge of the patient.

In another aspect, the dental implant system includes a plurality of holes defined by the prosthodontic base wherein the holes are positioned in part based on a cast of the edentulous ridge based on the impression and/or a radiography image of the alveolar bone of the patient.

In a further aspect of the invention, a method of providing permanent teeth replacement for a patient is disclosed. The method including obtaining an impression (conventional or digital) of the edentulous ridge of a patient wherein one or more teeth replacement are desired; forming a cast of the edentulous ridge based on the impression; and forming a prosthodontic base from the cast. The prosthodontic base defining first and second opposing surfaces. The method including shaping the first surface of the prosthodontic base to define a contour corresponding to the edentulous ridge, forming a post extending outwardly from the second surface, and cutting at least one hole through the prosthodontic base for attaching the prosthodontic base to the edentulous ridge. The method also includes forming a restoration including at least one replacement tooth, the restoration defining at least one opening for receiving the post(s) and articulating the restoration which may include attaching the prosthodontic base to the edentulous ridge of the patient and attaching the restoration to the prosthodontic base.

In another aspect, the disclosed method includes attaching the prosthodontic base on an edentulous ridge of the patient via a dental implant (or implants) inserted through the hole(s) in the prosthodontic base. In one embodiment, the step of installing the implant includes installing the implant using an automatic device such as a nail gun or a similar instrument.

In another aspect, the method includes positioning the at least one hole in the prosthodontic base based in part on a review of a radiography image of the edentulous ridge of the patient.

Another aspect of the disclosed method of providing permanent teeth replacement for a patient includes forming a prosthodontic base via a CAD/CAM process. The method of forming the prosthodontic base further includes forming a first surface of the prosthodontic base for abutment with the edentulous ridge of a patient from a tissue compatible material. The first surface comprising a contour made from a cast or graphical representation of the edentulous ridge so that the first surface fits thereon in a stable position in a custom-fit relation.

In another aspect, the method disclosed includes providing a permanent teeth restoration and attaching the same to the edentulous ridge of a patient without requiring oral surgery or a surgical incision of the oral mucous. The method including permanently attaching the prosthodontic base for engagement with and support on the edentulous ridge of the patient by installing implants through the prosthodontic base and into the edentulous ridge of the patient via an automatic implant installation device and without requiring surgical incision of the oral mucous. The method further including attaching the restoration to the prosthodontic base by adhering the restoration to at least one post extending outwardly from the prosthodontic base.

Since the dental implant system includes a prosthodontic base attachable to the edentulous ridge via mini-implants, the system can be installed in a dental office quickly and easily without requiring oral surgery. The dental implant system is installed on the edentulous ridge without requiring surgical incision of the oral mucous or suturing the same following the installation process.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The present teachings will become more fully understood from the detailed description, the appended claims and the following drawings. The drawings are for illustrative purposes only and are not intended to limit the scope of the present disclosure.

FIG. 1 is an illustration of one embodiment of a dental implant system in accordance with the present invention shown in use attached to the mandible of a patient.

FIG. 2 is a perspective view of one embodiment of a restoration according to the present invention dental implant system.

FIG. 3 is a perspective view of one embodiment of a prosthodontic base in accordance with the present invention.

FIG. 4 is a bottom side view of the prosthodontic base of FIG. 3.

FIG. 5 is a perspective view of a lower arch of a patient showing a prosthodontic base according to the present invention positioned thereon.

FIG. 6A-6C are side elevational views of various embodiments of dental implants in accordance with the present invention.

FIG. 7 is front side view of a lower arch of a patient shown with a prosthodontic base according to the present invention attached thereto.

FIG. 8 is a perspective view of a lower arch of a patient shown with a prosthodontic base according to the present invention positioned thereon.

FIG. 9 is a cross sectional of a mandible bone of a patient showing one embodiment of a prosthodontic base according to the present invention attached thereto.

### DETAILED DESCRIPTION OF EMBODIMENTS OF THE INVENTION

Detailed illustrative descriptions of example embodiments are disclosed herein. However, specific structural and functional details disclosed herein are merely representative for purposes of describing example embodiments. The example embodiments may be embodied in many alternate forms and should not be construed as limited to only the example embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or” includes any and all combinations of one or more of the associated listed items.

It will be understood that when an element is referred to as being “connected,” “coupled,” “mated,” “attached,” or “fixed” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between”, “adjacent” versus “directly adjacent”, etc.).

The terminology used herein is for the purpose of describing particular embodiments only and is not intended to be limiting of example embodiments. As used herein, the singular forms “a”, “an” and “the” are intended to include the plural forms as well, unless the language explicitly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

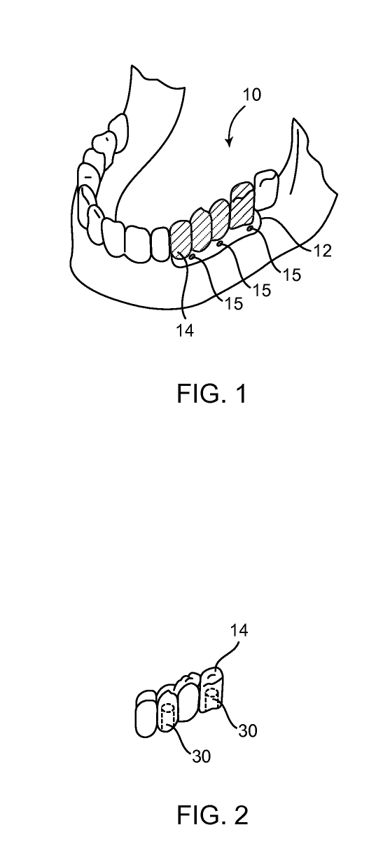

FIGS. 1-6 illustrate an example embodiment dental implant system 10 according to the present invention. The dental implant system 10 includes a prosthodontic base 12 and a restoration 14. The dental implant system 10 also includes a plurality of implants 15 which attach the prosthodontic base 12 to a patient's edentulous ridge 25.

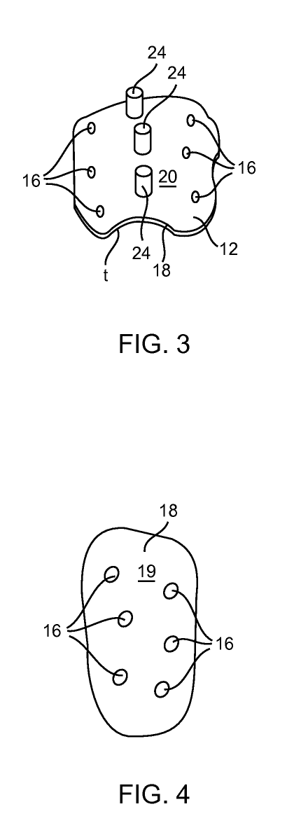

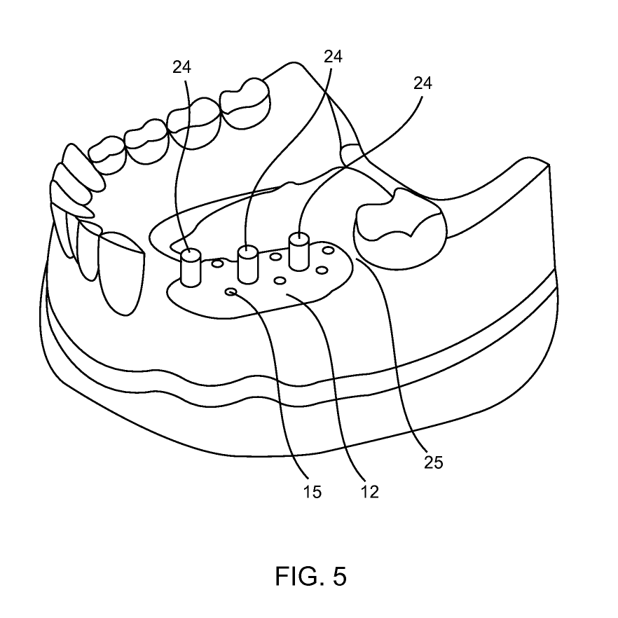

FIGS. 3 and 4 are illustrations of one embodiment of a prosthodontic. base 12 of the dental implant system 10. The prosthodontic base 12 is designed for mounting on the edentulous ridge 25 (See FIG. 5) of a patient (not shown) wherein teeth replacement is desired. The prosthodontic base 12 defines first surface and second opposing surfaces 18, 20 respectively. The first surface 18 defines a contour 19 shaped to conform to an edentulous ridge 25 of a patient wherein the prosthodontic base 18 is to be mounted. The second surface 20 is opposite the first surface 18 and located on a top side of the prosthodontic base 12. One or more posts 24 extend outwardly from the second surface 20 of the prosthodontic base 12 In the illustrated embodiment, the posts 24 are cylindrical and formed of the same material and integral with the prosthodontic base 12. In other embodiments, posts of different shapes can be used. Also, in other embodiments, the posts 24 can be formed separately from the prosthodontic base 12 and attached to the prosthodontic base using a fastener, adhesive or the like.

The contour 19 on the first surface 18 is shaped to conform to, and abut an edentulous ridge 25 of the patient (not shown). The first surface 18 is formed from a tissue-friendly material including one of gold, titanium, ceramic and/or alloys or combinations of thereof or similar tissue friendly materials. The prosthodontic base 12 may formed of one or more materials including wherein the first surface 18 is formed from a tissue friendly material such as those mentioned above, and a remainder of the prosthodontic base is formed from a different one of the materials mentioned above or another type of material such as plastic, acrylic or another durable material.

In one embodiment, the contour 19 of the prosthodontic base 12 is formed based on an impression taken of the edentulous ridge 25 of the patient by a dental professional. Bite registration information can be obtained conventionally or digitally. In a process well known in dentistry, the impression is then used to make a cast which is a replica of the edentulous ridge 25. The first surface 18 and contour 19 thereof is formed using the cast so that the contour 19 of the prosthodontic base 112 corresponds with the edentulous ridge 25 of the patient and is custom shaped to fit precisely thereon. Accordingly, the prosthodontic base 12 is custom manufactured to fit the edentulous ridge 25 of the patient. The position, orientation and size of the posts can be verified by the clinician as may be necessary. A temporary restoration (not shown) can also be made and provided to be used by the patient for testing the bite and/or during a time a permanent restoration is designed and manufactured.

Alternatively, the first surface 18 of the prosthodontic base 12 can be formed based on a digital impression of the edentulous ridge 25 of the patient. Bite registration information can also be obtained digitally.

In the embodiment of FIGS. 3 and 4, the prosthodontic base 12 is designed for a restoration 14 to replace a few teeth. However, in other embodiments, the prosthodontic base 12 can be configured to replace a single tooth, multiple teeth, or an entire arch of a patient's teeth.

Still referring to FIGS. 3 and 4, the prosthodontic base 12 defines a plurality of holes 16 which extend through a thickness t of the prosthodontic base. The holes 16 are sized to correspond to a size of an implant 15 and are positioned for securing the prosthodontic base 12 to the edentulous ridge of the patient. As discussed above, the prosthodontic base 12 is formed in part of a tissue friendly material for positioning the prosthodontic base directly on edentulous ridge 25 of the patient. Thus, there is no need for a surgical incision to sever the oral mucous and attach the prosthodontic base 12 directly to the alveolar bone of the patient such as with some prior art attachment procedures.

In one embodiment, the holes 16 are located based in part on a visual inspection of the edentulous ridge 25 by a clinician. A review of a radiography image of the edentulous ridge 25 can also be used to locate the holes 16 so that the implants 15 are positioned to securely attach the prosthodontic base 12 to the patient's bone. In another embodiment, a radiography image of the patient's bone structure and/or an image of the edentulous ridge 25 can be viewed with an overlay of an image of the proposed prosthodontic base 12 for locating the holes 16 in the prosthodontic base 12.

Alternatively, for patients with a healthy oral cavity and related bone structure, the holes 16 can be spaced apart along each of a lingual and buccal side of the prosthodontic base 12.

Referring to FIG. 5, on embodiment of a prosthodontic base 12 is shown mounted to the edentulous ridge 25 of a patient. The post(s) 24 are provided for attaching a restoration 14 to the prosthodontic base 12. The location and orientation of the post(s) 24 relative to the second surface 20 of the prosthodontic base 12 is determined based on a design of the restoration 14 proposed for mounting to the prosthodontic base. As shown in FIG. 2, the restoration 14 defines at least one opening 30 (e.g. a hole) for receiving the post(s) 24. The restoration 14 can be secured to the posts 24 via an adhesive attaching the posts 24 in the openings 30. The openings 30 are arranged to receive the posts 24 so that the restoration 14 is secured to the prosthodontic base 12 and articulated to correspond with the patient's opposing teeth. A coloration of the restoration 14 is also designed to correspond to the patient's teeth adjacent the restoration.

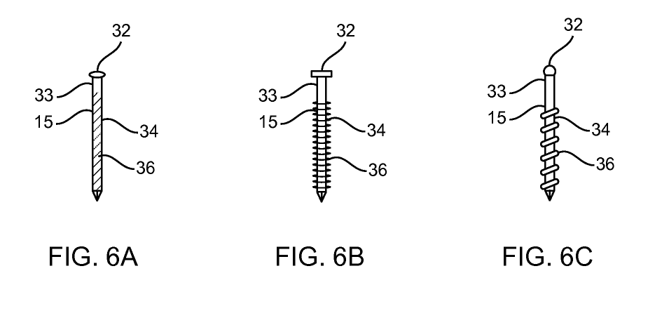

FIGS. 6A-6C are illustrations of various implants 15 in accordance with the present disclosure. The implants 15 be nail-shaped and include a head 32, neck 33, and body 34. The body 34 can include an engagement device 36 such as a threaded portion or other type surface characteristic used in conventional implants for securing the implant 15 to the patient's bone (not shown) and to promote osseointegration between the bone structure and the implant. In other embodiments, the implant 15 can be an endosteal implant defining pores (not shown) into which osteoblasts and supporting connective tissue can migrate.

The implants 15 can be configured for installation via a pneumatic, electric or other type of powered nail gun or installation device for efficient installation of the implants into the bone of a patient with minimum pain. Typically, the implants 15 are formed of titanium or an alloy thereof so that the implant is compatible with the bone and tissue of the patient. Depending on the application and the size and proposed location of the prosthodontic base 12, the implants 15 can be sized accordingly. The head 32 of the implant sized to be larger than the diameter of the holes 16 so the head draws the prosthodontic base 12 towards the edentulous ridge 25 and attaches the first surface 18 of the prosthodontic base to the tissue of the edentulous ridge. In one embodiment, the implant 15 has a diameter of about 1.6 mm and a length of about 8 mm. In other embodiments, the implant 15 has a diameter in a range between about 1.0 mm and 3.0 mm and a length in a range between about 6 mm to about 18 mm.

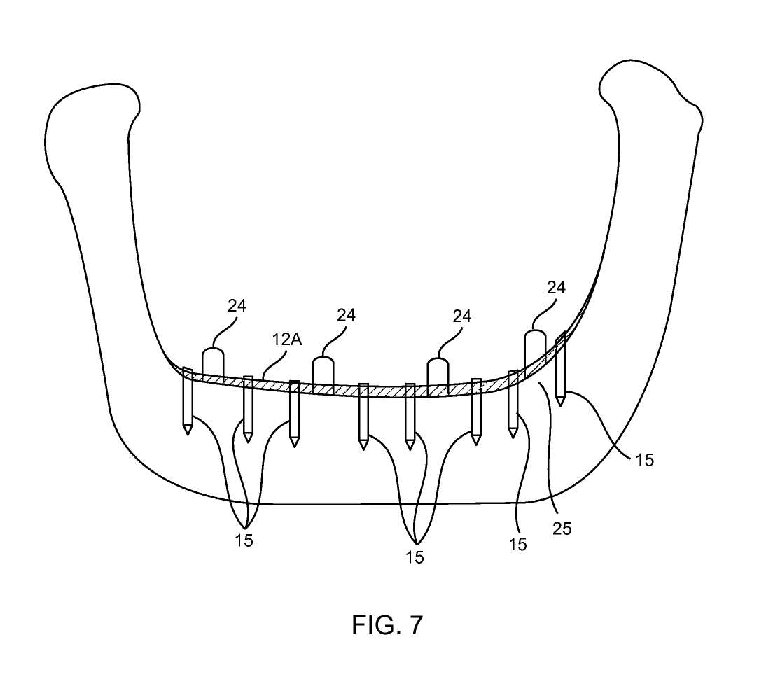

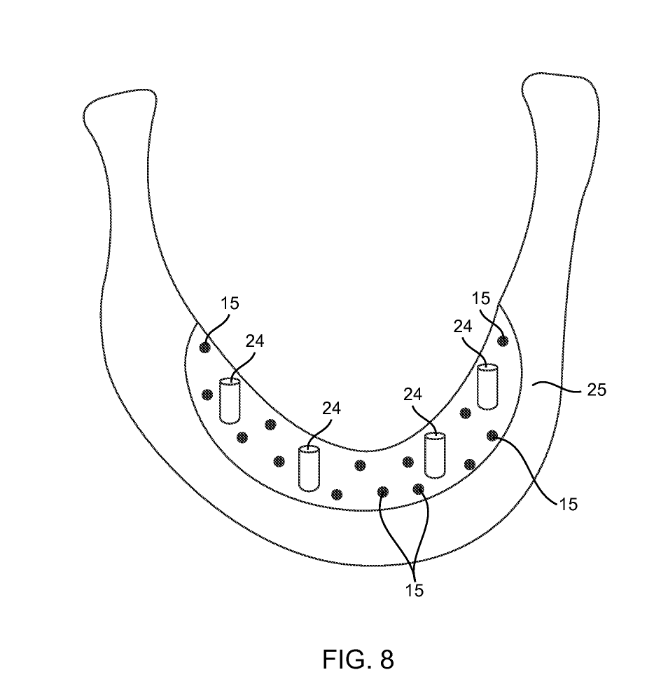

FIGS. 7 and 8 show a prosthodontic base 12A configured for replacing all of the teeth on the mandibular arch of an edentulous patient. The prosthodontic base 12A is shown attached to the mandibular bone 36 via a plurality of implants 15. The prosthodontic base 12A includes a plurality of posts 24 arranged for securing a restoration 14 to the prosthodontic base.

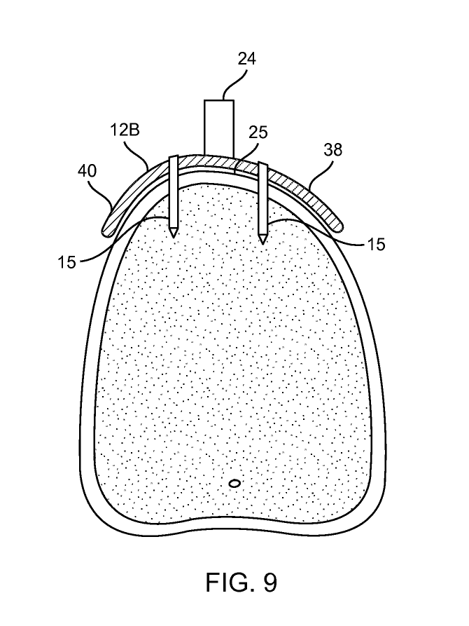

FIG. 9 is an illustration of another embodiment of a prosthodontic base 12B attached to the mandibular arch of a patient. As shown, the entire first surface 18 of the prosthodontic base 12B including the lingual and buccal edges 38, 40 thereof respectively, are formed to conform to and abut the edentulous ridge 25.

Also disclosed is a method of providing one or more permanent replacement teeth for a patient. The method including obtaining an impression (conventional or digital) of an edentulous ridge 25 of a patient wherein one or more teeth replacement are desired; forming a cast of the edentulous ridge based on the impression; and forming a prosthodontic base from the cast. The prosthodontic base defining first and second opposing surfaces. The step of forming a prosthodontic base including: shaping the first surface of the prosthodontic base 12 to define a contour 19 corresponding to the edentulous ridge 25 wherein the restoration is to be located, forming a post 24 extending outwardly from the second surface, and cutting or otherwise providing at least one hole 16 through the prosthodontic base for attaching the prosthodontic base to the edentulous ridge 25. The method further including forming a restoration 14 including at least one replacement tooth, the restoration defining one or more opening 30 for receiving the post/posts 24.

A step of articulating the restoration 14 including positioning and orienting the post 24 to extend outwardly from the second surface 20 of the prosthodontic base 12 so that the restoration fits in the patient's dentition adjacent any remaining teeth and conforms to the bite registration of the patient's opposing teeth. Additionally, the step of articulating the restoration includes positioning and orienting the opening(s) 30 defined by the restoration 14 to cooperate with the post(s) 24 so that the restoration 14 is positionable and mountable on the prosthodontic base 12 so that the restoration fits together with and the bite registration matches the adjacent or opposing teeth of the patient.

In one embodiment, the method further includes attaching the prosthodontic base 12 on an edentulous ridge 25 of the patient via an implant 15 inserted through the hole(s) 16 in the prosthodontic base. The implant 15 typically being a mini implant having a body diameter in a range between about 1.0 mm and about 2.9 mm. The step of inserting the implant through the prosthodontic base 12 and into the edentulous ridge 25 of the patient may include using an automatic device such as an electric or pneumatic nail gun to insert a nail shaped implant 15 into the bone of the patient. The process being a non-surgical process which can be performed by a dentist. The process of installing one or more implants 15 into the alveolar bone of the patient does not require a surgical incision of the tissue of the oral mucous or exposing the alveolar bone of the patient.

The method of forming the prosthodontic base 12 includes in one embodiment positioning at least one hole in the prosthodontic base based in part on a review of an radiography image of the alveolar bone of the edentulous ridge 25 of the patient. A visual inspection of the edentulous ridge 25 or a photo or image thereof may also be reviewed prior to locating the holes 16 for receiving the implants 15. This step helps to ensure the prosthodontic base 12 is secured to healthy tissue and bone of the patient for providing a permanent installation of the prosthodontic base.

In one embodiment, the method includes forming the prosthodontic base 12 in part via a CAD/CAM process wherein data from a digital scan of the edentulous ridge 25 of the patient is transmitted to a processor for use by a CAD device to design the prosthodontic bases 12 and corresponding restoration 14. An associated computer-aided machining (CAM) process can also be utilized for manufacturing and/or milling the prosthodontic base 12 and restoration 14 in accordance with the CAD designed restoration and prosthodontic base. The CAD/CAM process can be fully automated or require some inputs and interaction by an operator or technician operating the CAD/CAM device(s).

In one embodiment, one or more images of the patient's dentition, the edentulous ridge 25, and/or a radiography image or other type image of the patient's oral cavity including the edentulous ridge and/or associated teeth are used in one or more overlays in connection with the process of forming the prosthodontic base 12 and restoration 14.

The step of forming the prosthodontic base 12 further includes forming the first surface from a tissue compatible material which may include one of titanium, gold, porcelain, ceramic or another material which is compatible and non-toxic for abutment and long term positioning of the prosthodontic base 12 adjacent the edentulous ridge 25 and tissue of the patient.

The method further comprising attaching the prosthodontic base 12 to the edentulous ridge 25 via an implant 15 installed through the oral mucous of the patient as discussed hereinabove.

Example embodiments and methods thus being described, it will be appreciated by one skilled in the art that example embodiments and example methods may be varied through routine experimentation and without further inventive activity. Variations are not to be regarded as departure from the spirit and scope of the exemplary embodiments, and all such modifications as would be obvious to one skilled in the art are intended to be included within the scope of the following claims.

What is claimed is:

1. A dental implant system comprising:
a prosthodontic base having first and second opposing surfaces, the first surface configured to conform to and abut an edentulous ridge of a patient, the second surface having at least one post extending outwardly therefrom, the prosthodontic base defining at least one hole for receiving an implant;
a restoration comprising at least one artificial tooth, the restoration defining an opening to receive the post, the restoration being attachable to the post;
an implant having a head and a body, the body of the implant insertable through the hole in the prosthodontic base and into a bone of the patient for attaching the prosthodontic base to the edentulous ridge, the head having a diameter larger than a diameter of the hole for securing the prosthodontic base to the edentulous ridge; and wherein
the dental implant system provides for efficient, minimum invasive and permanent teeth replacement for the patient.

2. The dental implant system of claim 1 wherein the first surface of the prosthodontic base is formed based on an impression of the edentulous ridge.

3. The dental implant system of claim 1 wherein the first surface of the prosthodontic base is formed based in part on a digital scan of the edentulous ridge.

4. The dental implant system of claim 1 wherein the first surface of the prosthodontic base is formed based in part on a radiography image of the edentulous ridge.

5. The dental implant system of claim 1 wherein the implant is nail-shaped and configured for permanent attachment to the bone of the patient.

6. The dental implant system of claim 1 wherein the at least one hole defined by the prosthodontic base comprises a plurality of holes, the holes being positioned for locating the implant in a certain area of the bone of the patient identified in part based on a review of one of the digital scan and a radiography image of the edentulous ridge of the patient.

7. The dental implant system of claim 1 wherein the prosthodontic base is configured for permanent attachment to the edentulous ridge of the patient without requiring a surgical incision thereof.

8. The dental implant system of claim 1 wherein the prosthodontic base is formed in part from a tissue compatible material selected from one of gold, titanium, ceramic and porcelain.

9. The dental implant system of claim 1 wherein the restoration is configured to attach to the post via an adhesive.

10. The dental implant system of claim 1 wherein the implant is made of titanium.

11. The dental implant system of claim 1 wherein the implant is nail shaped and configured for installation via an automatic device.

12. The dental implant system of claim 1 wherein the body of the implant has a diameter less than 3.0 mm.

13. The dental implant system of claim 1 wherein the body of the implant is in a range between about 1.0 mm and about 2.9 mm.

14. A method of providing one or more replacement teeth for a patient, the method comprising:
obtaining an impression of an edentulous ridge of a patient wherein one or more teeth replacement are desired;
forming a cast of the foundation area based on the impression;
forming a prosthodontic base from the cast, the prosthodontic base defining first and second opposing surfaces, the step of forming a prosthodontic base including: shaping the first surface to define a contour corresponding to the edentulous ridge, forming a post extending outwardly from the second surface, and forming at least one hole through the prosthodontic base for attaching the prosthodontic base to the edentulous ridge; and
forming a restoration including at least one replacement tooth, the restoration defining an opening for receiving the post.

15. The method of claim 14 further comprising attaching the prosthodontic base on the edentulous ridge of the patient via a dental implant inserted through the hole in the prosthodontic base.

16. The method of claim 14 further comprising attaching the prosthodontic base on the edentulous ridge via a dental implant installed using an automatic device.

17. The method of claim 14 further comprising positioning the at least one hole in the prosthodontic base based in part on a review of an radiography image of the edentulous ridge.

18. The method of claim 14 wherein the step of forming the prosthodontic base is carried out in part via a CAD/CAM process.

19. The method of claim 14 wherein the step of forming the prosthodontic base further includes forming the first surface of a tissue compatible material.

20. The method of claim 14 further comprising attaching the dental base to the edentulous ridge via a dental implant installed through the oral mucous of the patient.
